(19) United States
(12) Patent Application Publication
(10) Pub. No.: US 2025/0089638 A1
(43) Pub. Date: Mar. 20, 2025

(54) INTERNET-OF-THINGS-BASED ALLERGEN POLLEN CONCENTRATION PREDICTION

(71) Applicant: INTERNATIONAL BUSINESS MACHINES CORPORATION, ARMONK, NY (US)

(72) Inventors: Li Li Guan, Beijing (CN); Zhe Yan, Beijing (CN); Rong Zhao, Beijing (CN); Li Bo Zhang, Beijing (CN); Hao Xiang Wu, Beijing (CN)

(21) Appl. No.: 18/469,665

(22) Filed: Sep. 19, 2023

(51) Int. Cl. A01H 1/00; A01H 1/04; G06V 20/10

(52) U.S. Cl. CPC A01H 1/121; A01H 1/04; G06V 20/188

(57) ABSTRACT

According to one embodiment, a method, computer system, and computer program product for pollen prediction is provided. The present invention may include identifying, by a classification model, plant species within a plurality of locatable images taken at a plurality of locations; creating growth cycle prediction models for the plant species; modelling pollen count mappings for the plant species; predicting pollen yields at the locations for the plant species based on the growth cycle prediction models and the pollen count mappings; and calculating a pollen distribution at the locations based on the predicted pollen yields and aerodynamic models.

### BACKGROUND

The present invention relates, generally, to the field of computing, and more particularly to environmental monitoring.

The field of environmental monitoring may be the field concerned with measuring and assessing levels of various substances present in the environment that could have an adverse effect on human health, such as allergens and pollutants. Environmental monitoring involves collecting meteorological data, topographical data, air quality data, soil quality data, and other such types of data from multiple sensors, and integrating these data types into digital models to understand the quality of the air or soil in a specific location or region. Environmental monitoring is essential for evaluating the potential impact of pollution and allergens on human health, the environment, and to support the development and implementation of air quality management strategies.

### SUMMARY

According to one embodiment, a method, computer system, and computer program product for pollen prediction is provided. The present invention may include identifying, by a classification model, plant species within a plurality of locatable images taken at a plurality of locations; creating growth cycle prediction models for the plant species; modelling pollen count mappings for the plant species; predicting pollen yields at the locations for the plant species based on the growth cycle prediction models and the pollen count mappings; and calculating a pollen distribution at the locations based on the predicted pollen yields and aerodynamic models.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a pollen concentration prediction process according to at least one embodiment;

FIG. 3 is an operational flowchart illustrating a pollen concentration prediction process according to at least one embodiment; and

FIG. 4 is a diagram illustrating an exemplary use case of a pollen concentration prediction process according to at least one embodiment.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to environmental monitoring. The following described exemplary embodiments provide a system, method, and program product to, among other things, determine a species distribution in a region, model the growth cycles of detected species, predict a pollen yield of the species based on the growth cycle, model a pollen distribution, and navigate a user based on the pollen distribution.

As previously described, the field of environmental monitoring may be the field concerned with measuring and assessing levels of various substances present in the environment that could have an adverse effect on human health, such as allergens and pollutants. Allergens are becoming an increasingly ubiquitous problem; the number of individuals suffering from seasonal allergies triggered by pollen has grown to encompass a significant portion of the global population, and this portion only grows year by year. As such, the need to monitor and track pollen levels in the air only increases with time.

Several attempts have been made in the art to address the issue of pollen monitoring. For example, one attempted solution includes deploying several pollen monitoring stations comprising air quality sensors spread within in a region to be monitored and generalizing a pollen level over the entire region. However, this solution only provides general information for larger regions or cities, and cannot support personalized management; because pollen concentrations can vary significantly over short distances and short timeframes due to factors such as wind patterns, topography, vegetation, and human activity, such general conclusions cannot capture the pollen levels at a granularity that is small enough to, for example, describe pollen levels on a street-by-street basis that would be useful in informing a user's navigation through a region. In order to provide granular enough data to capture pollen on a basis that could enable navigation, such a method would have to deploy hundreds, perhaps thousands, of additional monitoring stations, which would prove expensive, intrusive, and difficult.

Another attempted solution includes utilizing wearable allergen detection equipment worn on the person of the user and used to detect pollen levels in the environment around the user. However, such methods need to be in a certain environment to work. Traditional attempts at addressing the problem of pollen monitoring rely on manual sampling and specialized equipment, which can be costly and limited in number and coverage. Existing monitoring stations and pollen sensors may not be sufficient in number and coverage to achieve granularity small enough to enable navigation. Furthermore, traditional attempts lack real-time capabilities; because allergen concentrations may vary significantly over time and location, allergen concentrations may be difficult to monitor and predict, and measured pollen levels extrapolated to describe even a modestly sized area may often be inaccurate.

Other attempted solutions may include inferring the presence of pollen from information such as allergy medication usage relative to number of allergic individuals, allergic behaviors observed in individuals within a region, et cetera. Such attempted solutions cannot calculate allergen concentrations with any specificity or granularity, and furthermore cannot identify the presence of particular allergens originating from individual species or sub-species of plants.

As such, it may be advantageous to, among other things, implement a system that leverages existing Internet of Things (IoT) sensors, infrastructure, and environmental data in combination with a model of the growth rate of local plant species and sub-species of a region to measure, analyze, and predict allergen concentrations from those local species and subspecies in real time within that region, at a granularity small enough to enable navigation and without necessitating the deployment of additional equipment beyond what already exists in the region. Therefore, the present embodiment has the capacity to improve the technical field of environmental monitoring by providing a system that identifies allergen concentrations of individual species and sub-species of plants at a granular enough level to enable a user to navigate on a street-by-street basis to avoid allergen concentrations, and maintaining that granularity even where there is no coverage or only partial coverage by environmental monitoring stations and sensors, thereby providing a low-cost, high-accuracy method of pollen concentration modeling that can allow users to move about outside while safely avoiding the specific allergens to which they are allergic. In other words, the system may be capable of calculating and predicting allergen concentration with wider coverage and smaller granularity than existing methods using phenological data and existing common IoT data, which allows individuals to make informed decisions about their outdoor activities, including their daily errands, travel plans, or adventures. By leveraging existing IoT infrastructure, it becomes feasible to gather data from multiple sources and create a more detailed picture of allergen status in different areas. By utilizing existing IoT data, which is already being collected for various purposes, the system may improve resource allocation by avoiding the need for additional equipment or data collection processes, and by making use of readily available data streams. This efficiency can help reduce costs and ensure the sustainability of allergen monitoring initiatives.

According to at least one embodiment, the invention is a system that determines a species distribution in a region, models the growth cycles of detected species, predicts a pollen yield of the species based on the growth cycle, models a pollen distribution based on the pollen yield and an aerodynamic model, and navigates a user based on the pollen distribution.

According to at least one embodiment, the invention is a system that determines a species distribution in a plurality of regions, assigns a reference region for each plant species in the distribution from among the plurality of regions where the plant species is identified, trains a growth cycle prediction model for the plant species of the distribution, trains a deviation correction model on phenological data from the plurality of regions, predicts a pollen yield for each species in the regions based on the growth cycle prediction model and compensates for any discrepancies between the growth rate and growth cycle of plant species in the region by normalizing such differences between the reference region and other non-reference regions using the deviation correction model, models a pollen distribution based on the pollen yield and an aerodynamic model, and navigates a user based on the pollen distribution.

In embodiments, Internet of Things sensor data may be recorded from IoT devices, which may be existing devices deployed within the one or more regions. The system uses IoT sensor data to provide contextual data about the sensor's surroundings. The IoT sensor data may, among other things, be used in conjunction with growth cycle prediction models and the aerodynamics models to enable correction for deviation in growth cycle models and particle dispersion models in different environments and thus predict the correct pollen yield at different locations.

According to an aspect of the invention, there is a provided a method for pollen prediction comprising the steps of identifying, by a classification model, one or more plant species within a plurality of locatable images taken at a plurality of locations; creating one or more growth cycle prediction models for the one or more plant species; modelling one or more pollen count mappings for the one or more plant species; predicting one or more pollen yields at the locations for the one or more plant species based on the one or more growth cycle prediction models and the one or more pollen count mappings; and calculating a pollen distribution at the locations based on the one or more predicted pollen yields and one or more aerodynamic models. By making separate growth cycle prediction models for individual plant species, and inferring pollen yields at multiple individual locations, this aspect of the invention allows granular assessment of specific types of pollen on a per-species basis, at individual locations in a region.

In embodiments, the method may optionally comprise the steps of navigating a user based on the one or more pollen distributions at one or more of the locations. Navigating the user based on the pollen yield leverages the granular assessment and species granularity of the method to enable the user to navigate through a region while avoiding concentrations of pollen from specific plant species at individual locations. In such embodiments, the greater the number of locations, the more accurate the navigation.

In some embodiments, the locations may optionally be located within a plurality of regions. Such enables the method to cover a wider territory, spanning multiple geographical zones which may have differing environmental conditions, and enabling coverage of a wider area, which may improve the reach and accuracy of navigation.

In some embodiments, the method may optionally comprise the step of designating one of the regions as a reference region for a plant species of the one or more plant species, where the growth cycle prediction model for the plant species is created based on historical phenological data of the reference region. This designation of a reference region enables accurate initial assessment of the plant's growth cycle using a growth cycle prediction model for a single region, which can then be cheaply leveraged and modified to extrapolate the growth cycle for other regions.

In some embodiments, the invention may optionally comprise the step of creating one or more deviation correction models for the plant species based on historical phenological data pertaining to the one or more regions comprising the plant species that are not the reference region of the plant species. In some embodiments, the calculating step may optionally comprise the step of, responsive to determining that a location of the locations does not comprise the reference region, normalizing an output of the growth cycle prediction model pertaining to the plant species using a deviation correction model of the one or more deviation correction models pertaining to the plant species and a region of the regions comprising the location. This embodiment, in combination with the prior embodiment, enables the creation and operational deployment of the deviation correction models, which enable the invention to cheaply and easily modify the output of the growth cycle prediction model to apply to other regions.

In some embodiments, the calculating may optionally be performed based on a plurality of real-time Internet of Things sensor data. This enables the invention system to leverage real time sensors on the ground to improve the accuracy of inferences regarding the growth cycle and growth rate of plant species on the ground.

In an exemplary embodiment Amy, a woman who suffers from hay fever, is aware of her allergies to willow and acacia flowers, but existing pollen maps fail to provide detailed information. The pollen maps only offer a general overview of pollen concentrations in the area without distinguishing between specific allergens or providing localized data for different streets. As a result, Amy is often confined indoors, unable to enjoy outdoor activities or travel freely. However, utilizing the invention system, which comprises many nearby locations in turn comprising plant data such as street vegetation coverage, number of plants, and plant status information, Amy gains access to real-time and granular information about the concentrations of willow and acacia pollen on different streets at any given moment. Armed with this precise knowledge, Amy discovers that the 2nd route of her planned hiking trip, from Third Street to Century Square, is predicted to meet her allergy criteria by 3 PM. With this information, Amy embarks on her journey. The system empowers Amy to navigate the streets with awareness, knowing which areas are safe from high concentrations of willow and acacia pollen. While other allergens may still be present in the air, Amy can travel freely knowing that the specific allergens to which she is allergic are not present.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

The following described exemplary embodiments provide a system, method, and program product enabled to determine a species distribution in a region, model the growth cycles of detected species, predict a pollen yield of the species based on the growth cycle, model a pollen distribution, and navigate a user based on the pollen distribution.

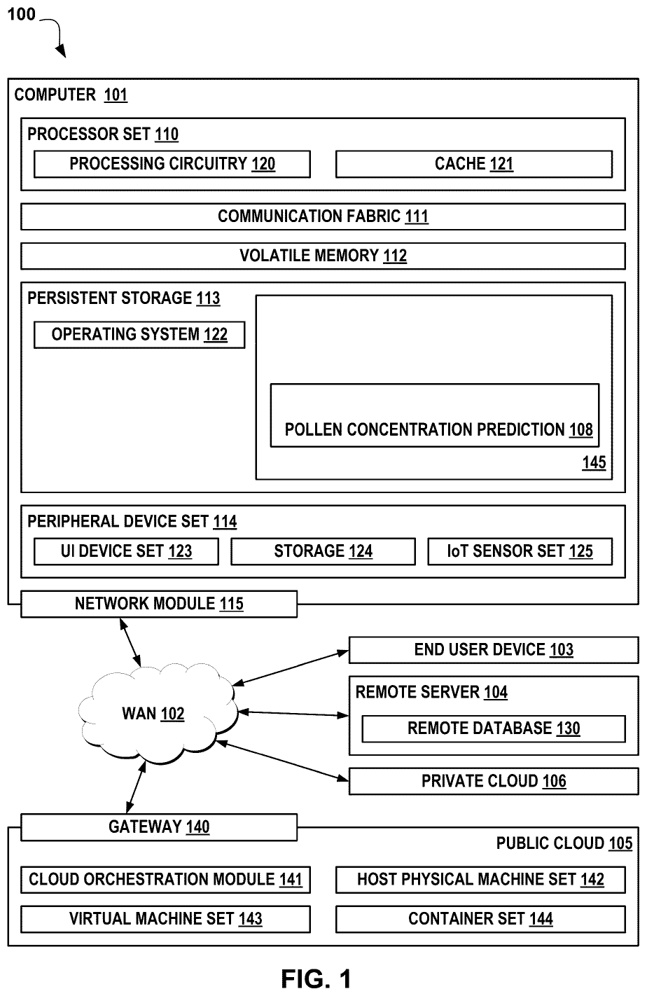

Referring now to FIG. 1, computing environment 100 contains an example of an environment for the execution of at least some of the computer code involved in performing the inventive methods, such as code block 145, which may comprise pollen concentration prediction program 108. In addition to code block 145, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and code block 145, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in code block 145 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface type operating systems that employ a kernel. The code included in code block 145 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. The term “Internet of Things” describes devices equipped with sensors such as those comprising IoT sensor set 125, processing means, and networking capabilities that connect and exchange data with other devices over a communications network. IoT sensor set 125 may be integrated into or in communication with such IoT devices and may be deployed at one or more locations within one or more regions so as to gather data regarding the environment and/or plant growth at the location. IoT sensor set 125 may comprise such sensors as temperature sensors, humidity sensors, solar collectors, photosensors, wind sensors, visual and infrared cameras, et cetera. The IoT sensor set 125 may record and relay data to the pollen concentration prediction program 108 in real time, to enable real-time assessment of growth rate, growth cycle, and environmental conditions at the location. The data recorded by IoT sensor set 125 may comprise meteorological data, which may comprise data regarding the local weather conditions such as precipitation, wind, light intensity, light duration, temperature change curve over time, temperature differences between night and day, humidity, et cetera, or may comprise plant data, which is data pertaining to the current health and state of plant species such as local atmospheric oxygen content, soil quality, time to reach optimal growth temperature, visual data including color, size, number of leaves, vegetation density, et cetera.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101) and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

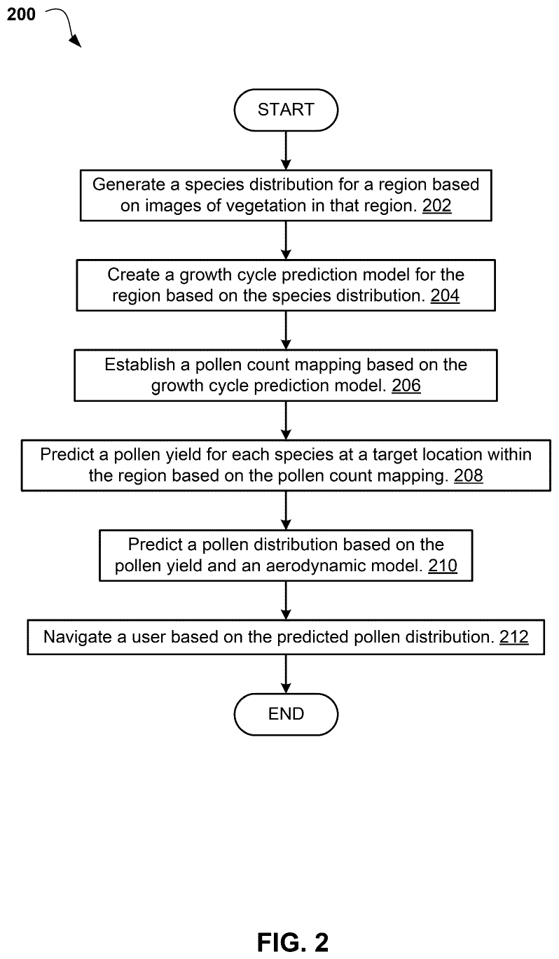

According to the present embodiment, the pollen concentration prediction program 108 may be a program capable of determine a species distribution in a region, model the growth cycles of detected species, predict a pollen yield of the species based on the growth cycle, model a pollen distribution, and navigate a user based on the pollen distribution. The pollen concentration prediction program 108 may, when executed, cause the computing environment 100 to carry out a pollen concentration prediction process 200. The pollen concentration prediction process 200 may be explained in further detail below with respect to FIG. 2. In embodiments of the invention, the pollen concentration prediction program 108 may be stored and/or run within or by any number or combination of devices including computer 101, end user device 103, remote server 104, private cloud 106, and/or public cloud 105, peripheral device set 114, and server 112 and/or on any other device connected to WAN 102. Furthermore, pollen concentration prediction program 108 may be distributed in its operation over any number or combination of the aforementioned devices.

Referring now to FIG. 2, an operational flowchart illustrating a pollen concentration prediction process 200 is depicted according to at least one embodiment. At 202, the pollen concentration prediction program 108 may generate a species distribution for a region based on images of vegetation in that region. Here, the pollen concentration prediction program 108 may determine a species distribution using a classification model that analyzes images of vegetation from specific locatable sources; in other words, images taken at or of a known location. For example, locatable sources may include satellite imaging data of known coordinates that include the region, camera footage from cameras deployed at known locations in the region or worn or carried by users in the region whose location is tracked by GPS, et cetera. The classification model may be a machine learning model that has been trained to identify specific species and sub-species of plants within an image of vegetation utilizing image processing techniques. The classification model may take as input the images and corresponding locations, and output all species found within the input images. In embodiments, the classification model may merely analyze the input images for plants that produce pollen that is known to produce allergic reactions in humans. The pollen concentration prediction program 108 may combine all found species into a single distribution, which represents a list of all plant species and subspecies in the region. A region may be a geographical zone which could range in size and character from a continent such as Asia or Africa, a country such as the United States or Australia, a biome such as a tropical rainforests or desert, or a specific ecosystem such as an alpine meadows or coastal wetland. A region may be a country, zip code, municipality, property, state, et cetera. In embodiments, the main defining trait of the region is that it experiences environmental conditions that differ significantly from adjacent regions. The environmental conditions of a region may differ significantly from those of an adjacent region where the growth cycle and/or growth rate of a plant species differ between the two regions by more than a threshold amount, which may be pre-supplied to the pollen concentration prediction program 108. In areas with highly localized weather patterns, such as the San Francisco Bay area where prevailing wind off the Pacific Ocean creates areas with permanent cloud cover, regions may be merely a few square miles; in areas with larger, more monolithic environmental conditions, such as the Sahara Desert, regions may cover hundreds or even thousands of square miles.

At 204, the pollen concentration prediction program 108 may create a growth cycle prediction model for the region based on the species distribution. Here, the pollen concentration prediction program 108 may retrieve data from a corpus of historical phenological data. Phenology relates to the study of plant and animal life cycles in relation to weather and climate; phenological data includes species, locations, life cycle, and environment data. The corpus may comprise a database of information regarding the growth rate and growth cycle of various plant species and sub-species, such as how fast a plant grows from seed to maturity, the various stages of growth that the plant may experience such as sprouting, budding, flowering, and ripening, and how environmental conditions such as soil composition, presence of and competition with other plants, meteorological conditions, et cetera affect the growth cycle and growth rate of the plant; for example, a plant's growth rate, the growth cycle the plant experiences and the plant's progress through various stages of its growth cycle may change depending on the temperatures and the respective amounts of water, sun, wind, nutrients, et cetera that the plant is exposed to. The corpus may further comprise information regarding how to infer a stage of growth of a given plant; for example, the corpus may comprise information regarding how to infer a stage of growth of a plant from visual characteristics that could, for example, be extracted from the locatable images, which may include a plant's height, the presence and/or size of buds or flowers or fruits on the plant, the size or number of leaves, the plant's color, the presence, density, current growth cycle and growth rate of competitor plants, et cetera. The corpus may comprise information regarding how to infer a stage of growth of a plant from local or regional environmental conditions that could, for example, be extracted from data measured by IoT sensor data and/or from meteorological data, such as local oxygen content, light intensity, wind patterns, soil composition, temperature, rainfall, time of year, et cetera. The corpus may further comprise cyclical changes in the environment of a given region such as seasonal weather, rainfall, temperature, sunlight, et cetera, and/or how the changes influence local plant species. The historical phenological data may be pre-provided, and/or stored in a local data repository, for example within storage 124 or persistent storage 113, and/or may be located remotely on remote server 104 or private cloud 106.

The pollen concentration prediction program 108 may retrieve data from the corpus of historical phenological data pertaining to each of the plant species and/or sub-species present within the distribution. The pollen concentration prediction program 108 may train a growth cycle prediction model on the retrieved historical phenological data for the plant species and sub-species. In some embodiments, the pollen concentration prediction program 108 may train the growth cycle prediction model on environmental conditions present in the region. The growth cycle prediction model may be a machine learning model that, once trained on the historical phenological data for the plant species and sub-species of the region, can receive as input a target day, plant data for a given plant at a target location, and environmental data for the target location and/or the region, and can output predicted values for the growth rate and growth cycle of the target plant on the target date. The growth cycle value may, for instance, comprise the growth stage that the plant is currently experiencing, and the number of days since the plant originally entered that growth stage. The target locations may be discrete geographical points or sub-zones of a region which are located within areas that are visually depicted by one or more of the locatable images, and/or are within a threshold distance of a location associated with one or more of the locatable images, and/or are within a threshold distance of an IoT sensor. The threshold distance may represent a physical distance from a locatable image or IoT sensor within which conditions or information extracted from the image or recorded from the IoT sensor could still reasonably apply to a pollen yield prediction without loss of accuracy. The target date may be the day for which the pollen concentration prediction program 108 is attempting to predict pollen concentration, which may be the current date or a past date. In an example, once the training is complete, pollen concentration prediction program 108 may provide a target date, plant data regarding a given plant, including visual information regarding the target plant and competitor plants extracted from the one or more locatable images, and environmental data including meteorological data and/or IoT sensor data pertaining to, for example, temperature, humidity, wind, rainfall, light, cloud cover, et cetera, as inputs to the growth cycle prediction model. The pollen concentration prediction program 108 may then receive a growth rate and predicted point or stage that the target plant has reached in its growth cycle as of the target date as outputs from the growth cycle prediction model. Growth rate may be the absolute growth of a plant species per unit of time, and the growth cycle may be the current growth stage that the plant species has reached and the day of that growth stage.

In embodiments, the target date may be a date in the future; in such embodiments, the pollen concentration prediction program 108 may provide environmental data comprising forecasted meteorological data or estimated meteorological data extrapolated from historical trends, as opposed to observed meteorological data, to the growth cycle prediction model to extrapolate a growth cycle and growth rate from the historical phenological data.

At 206, the pollen concentration prediction program 108 may establish a pollen count mapping based on the growth cycle prediction model. Here, the pollen concentration prediction program 108 may create a mapping between the pollen count of a given species or sub-species of plant and its growth rate and growth cycle. To put another way, any given plant species or sub-species generates pollen in differing amounts at different points in its growth cycle. For example, an Athelas plant may enter its flowering stage once it has reached maturity; upon entering the flowering stage, the plant may initially produce increasing amounts of pollen each day, until an inflection point where plant produces less pollen each day until the flowering stage of the plant's life cycle has concluded. The point at which a given plant reaches maturity and enters into the flowering stage, the time spent in the flowering stage and the daily pollen production that the plant achieves at each day of the flowering stage, et cetera may all be present in and extracted from the historical phenological data. As such, the pollen concentration prediction program 108 may retrieve historical phenological data from the corpus for each plant species or sub-species identified in the species distribution, and may analyze the historical phenological data for that plant species or sub-species to extract the relationship between the plant species' growth rate, the specific day within the plant's growth cycle, and the amount of pollen that the plant produces under conditions historically present in the associated region. This extracted relationship may be stored as a pollen count mapping.

At 208, the pollen concentration prediction program 108 may predict a pollen yield for each species at a target location within the region based on the pollen count mapping. Here, the pollen concentration prediction program 108 may predict the pollen yield at the target location by calculating a pollen count for each target plant, where the target plant may comprise all, or some plant or plant species present at the target location. The pollen concentration prediction program 108 may calculate a pollen count for a target plant by providing the target date, plant data regarding the target plant and environmental data for the region as inputs to the growth cycle prediction model and receiving the growth cycle and growth rate of the target day back as an output. The pollen concentration prediction program 108 may retrieve a pollen count mapping for the target plant and may compare the growth cycle and growth rate of target plant against the pollen count mapping to obtain the pollen yield for the target plant, or the amount of pollen produced by the target plant on the target day.

In embodiments, the pollen concentration prediction program 108 may obtain the pollen yield for a target plant by calculating a pollen concentration for the target date and each day prior up to a start date, and add together the amount of pollen produced each day such that the pollen yield represents an aggregate quantity comprising the sum of all pollen produced by the target plant between the start date and the target date. The start date may be a day one or more days prior to the target date; in embodiments, the start date may be a number of days prior to the start date selected based on the historical phenological data to represent the window of time beyond which pollen produced by the target plant would dissipate from a target location, such that all pollen produced by the target plant within that window of time would accumulate at the location. In embodiments, for example where the pollen concentration prediction program 108 has identified the target plant as being within a growth cycle associated with pollen production, the start date may be the day at which the plant first entered the growth cycle associated with pollen production, based on the growth cycle and the growth stage of the target plant.

In embodiments, for example where a user is navigating based on the pollen yield prediction, the target location may additionally be a location where the user wishes to navigate, the target date may comprise the current date or a date that the user has selected as the day when the user wishes to navigate to the target location. In embodiments, the target plant may be a species or sub-species of plant which the user has selected for pollen yield prediction, and/or may be plant species or sub-species to which the user is allergic, as evidenced by, for example, pre-provided information in a user profile accessible to pollen concentration prediction program 108, or a user's response entered into a prompt provided by pollen concentration prediction program 108 and requesting information regarding the user's allergies. In embodiments, pollen concentration prediction program 108 may only calculate pollen yield for the species and/or sub-species of plant to which the user is allergic.

At 210, the pollen concentration prediction program 108 may predict a pollen distribution based on the pollen yield and an aerodynamic model. Here, the aerodynamic model may be a machine learning model which may be trained to model the airborne flow and diffusion of particulates from an origin point, and which provides a particle diffusion coefficient for a target location at a target date given the environmental diffusion conditions around the target location at the target date. The particle diffusion coefficient may represent the average rate at which pollen diffuses outwards from the location where it originated. The pollen concentration prediction program 108 may accordingly extract environmental diffusion conditions from a variety of real-time IoT sensor data and/or meteorological data including, for example, wind speed and direction, vegetation density, humidity, et cetera. In embodiments, the aerodynamic model may further accept topographical information as input, and pollen concentration prediction program 108 may extract topographical information using locatable images of or taken at or near the location to identify topographic features such as buildings, trees, terrain features, et cetera. The pollen concentration prediction program 108 may provide the extracted environmental diffusion conditions, and in embodiments, topographical information, pertaining to the target location at the target date, to the aerodynamic model as inputs to receive the particle diffusion coefficient as an output. In embodiments, the pollen concentration prediction program 108 may provide environmental diffusion conditions pertaining to a period of time between a start date and the target date as inputs to the aerodynamic model, and may receive as output a particle diffusion coefficient that pertains to the period between the start date and the target date, and represents the average daily rate at which pollen diffuses outwards from the target location in the period of time between the start date and the target date. The pollen concentration prediction program 108 may modify the pollen yield of the target plants by the particle diffusion coefficient to predict a pollen distribution for the target plant, where the pollen distribution represents the actual amount of pollen from each target plant which is present at the target location.

At 212, the pollen concentration prediction program 108 may navigate a user based on the predicted pollen distribution. Here, the pollen concentration prediction program 108 may provide instructions to a user to navigate within the region and through or around at least one target location based on the pollen concentration of target plants at the target location on the target date. The pollen concentration prediction program 108 may display a graphical user interface to the user on the user device 103 or display of UI device set 123 of computer 101; the graphical user interface may, for example, prompt the user to enter a target destination, target time, and one or more target plant species or sub-species to which the user may be allergic or otherwise wish to avoid. The pollen concentration prediction program 108 may then, utilizing an online mapping process, derive a route from the user or from a user-specified starting location to the target location at the target time that avoids locations containing the target plant species or sub-species, and/or avoids locations containing pollen concentrations from the target plant species or sub-species that exceed a threshold value, where the threshold value represents a pollen concentration sufficient to trigger allergic symptoms or allergic symptoms of a particular severity in the user. The threshold value may be pre-provided, entered by the user, and/or derived by a machine learning model based on historical data.

In embodiments, rather than or in addition to navigating the user, the pollen concentration prediction program 108 may provide alerts or notifications to the user when the pollen concentrations of the target plant species at the user's location exceed the threshold value, or at times in the future when the target plant species at the user's location will be predicted to exceed the threshold value.

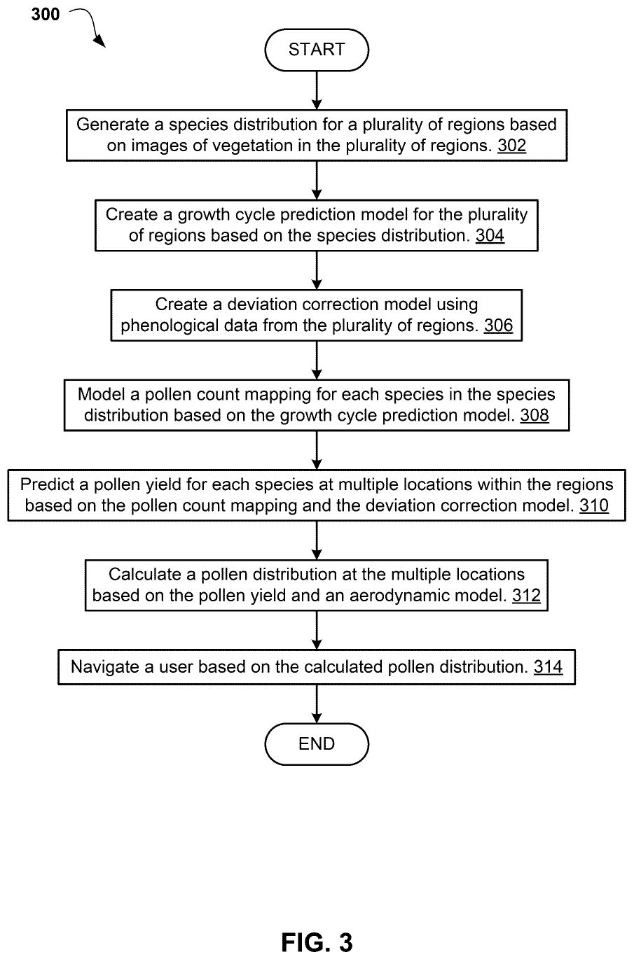

Referring now to FIG. 3, an operational flowchart illustrating a pollen concentration prediction process 300 is depicted according to at least one embodiment. At 302, the pollen concentration prediction program 108 may generate a species distribution for a plurality of regions based on images of vegetation in each region. Here, the pollen concentration prediction program 108 may determine a species distribution using a classification model that analyzes images of vegetation from specific locatable sources. The classification model may be a machine learning model that has been trained to identify specific species and sub-species of plants within an image of vegetation. The classification model may take as input the images and corresponding locations, and output all species found within the input images. In embodiments, the classification model may merely analyze the input images for plants that produce pollen that is known to produce allergic reactions in humans. The pollen concentration prediction program 108 may combine all found species into a single species distribution, which represents a list of all plant species and subspecies in all of the regions. In embodiments, the pollen concentration prediction program 108 may create a species distribution for each region, where each species distribution comprises all plant species and sub-species identified in locatable images taken in a given region.

The regions may be separate geographical zones which could range in size and character from continents such as Asia or Africa, countries such as the United States or Australia, biomes such as tropical rainforests or deserts, or specific ecosystems such as alpine meadows or coastal wetlands. Regions may be counties, zip codes, municipalities, properties, states, et cetera. There may be differences in climate or other environmental conditions between regions, such as wind or weather patterns, which may alter the growth cycles of plants within one region relative to those of the same species in another region.

At 304, the pollen concentration prediction program 108 may create a growth cycle prediction model for the plurality of regions based on the species distribution. Here, the pollen concentration prediction program 108 may retrieve data from a corpus of historical phenological data. The corpus may comprise a database of information regarding the growth rate and growth cycle of various plant species, as well as the cyclical changes in the environment of the reference region, and how the environment influences the plant species. The historical phenological data may be pre-provided, and/or stored in a local data repository, for example within storage 124 or persistent storage 113, and/or may be located remotely on remote server 104 or private cloud 106. The pollen concentration prediction program 108 may assign each plant species or subspecies from the distribution or distributions an associated reference region; the reference region may be a region of the plurality of regions where the plant species or subspecies has been identified, and for which pollen concentration prediction program 108 will train a growth cycle prediction model on historical phenological data for that plant species or sub-species relative to the historical environmental conditions of the reference region; the differences between one region and another region may be significant enough that a growth cycle prediction model created for one region may be inaccurate when applied to another region, but because such differences can be easily identified and normalized for, creating a growth cycle prediction model for every region is expensive and unnecessary.

For each of the plant species and/or sub-species present within the distributions, the pollen concentration prediction program 108 retrieves data from the corpus of historical phenological data pertaining to the plant species or sub-species and its assigned reference region. The pollen concentration prediction program 108 then trains a growth cycle prediction model on the retrieved historical phenological data for the plant species and sub-species relative to the historical environmental conditions of the reference region. The growth cycle prediction model may be a machine learning model that, once trained on the historical phenological data for the plant species and sub-species with respect to their assigned reference regions, can receive as input a target day, plant data for a given plant at a target location, and environmental data for the target location and/or the region, and can output predicted values for the growth rate and growth cycle of the target plant on the target date. In an example, once the training is complete, pollen concentration prediction program 108 may provide a target date, plant data regarding a given plant, including visual information regarding the target plant and competitor plants extracted from the one or more locatable images, and environmental data including meteorological data and/or IoT sensor data pertaining to, for example, temperature, humidity, wind, rainfall, light, cloud cover, et cetera of the target location and/or reference region, as inputs to the growth cycle prediction model. The pollen concentration prediction program 108 may then receive a growth rate and predicted point or stage that the target plant has reached in its growth cycle as of the target date as outputs from the growth cycle prediction model.

At 306, the pollen concentration prediction program 108 may create a deviation correction model using the phenological data from the plurality of regions. The growth cycle prediction model is used as a base model for the deviation correction model, but whereas the growth cycle prediction model is used as one species' or sub-species' base model and is trained on environmental conditions within the reference region, the deviation correction model is trained on historical phenological data of the same species or sub-species under different environmental conditions in the multiple regions. The resultant deviation correction models, once trained, can be used to normalize the predicted values of growth rate and growth cycles based on IoT data and climate data from different regions, by modifying the predicted values to represent the changes to a plant's growth rate or growth cycle based on differing environmental conditions between the reference region and another region as derived from the IoT data and climate data. The pollen concentration prediction program 108 may create a deviation correction model for each region for each plant species and/or sub-species present in multiple regions, except for the reference region of those plant species and/or sub-species.

At 308, the pollen concentration prediction program 108 may model a pollen count mapping for each species in the species distribution based on the growth cycle prediction model. Here, the pollen concentration prediction program 108 may create a mapping between the pollen count of a given species or sub-species of plant and its growth rate and growth cycle. To put another way, any given plant species or sub-species generates pollen in differing amounts at different points in its growth cycle. As such, the pollen concentration prediction program 108 may retrieve the historical phenological data for each plant species identified in the species distribution, and may analyze the historical phenological data for that plant species to extract the relationship between the plant species' growth rate, the specific day within the plant's growth cycle, and the amount of pollen that the plant produces under conditions historically present in the associated region. This extracted relationship may be stored as a pollen count mapping.

At 310, the pollen concentration prediction program 108 may predict a pollen yield for each species at multiple locations within the regions based on the pollen count mapping and the deviation correction model. Here, the pollen concentration prediction program 108 may predict the pollen yield at the target locations by calculating a pollen count for each target plant. The pollen concentration prediction program 108 may calculate a pollen count for a target plant at a target location by providing the target date, plant data regarding the target plant and environmental data for the region as inputs to the growth cycle prediction model, and receiving the growth cycle and growth rate of the target plant on the target day at the target location back as an output. For each target plant at each target location, the pollen concentration prediction program 108 may determine whether the target location is located in the reference region of the target plant. If the target location is not located in the reference region associated with the target plant, the pollen concentration prediction program 108 may provide the growth cycle and growth rate of the target plant received from the growth cycle prediction model as inputs to a deviation correction model pertaining to the target plant in the region comprising the target location; the pollen concentration prediction program 108 may then receive values for the growth cycle and growth rate back from the deviation correction model that have been normalized to account for the differences between the reference region and the region comprising the target location. The pollen concentration prediction program 108 may retrieve a pollen count mapping for the target plant, and may compare the normalized growth cycle and normalized growth rate of the target plant against the pollen count mapping to obtain the pollen yield for the target plant, or the amount of pollen produced by the target plant on the target day.

In embodiments, the pollen concentration prediction program 108 may obtain the pollen yield for a target plant by calculating a pollen concentration for the target date and each day prior up to a start date, and add together the amount of pollen produced each day such that the pollen yield represents an aggregate quantity comprising the sum of all pollen produced by the target plant between the start date and the target date. The start date may be a day one or more days prior to the target date; in embodiments, the start date may be a number of days prior to the start date selected based on the historical phenological data to represent the window of time beyond which pollen produced by the target plant would dissipate from a target location, such that all pollen produced by the target plant within that window of time would accumulate at the location. In embodiments, for example where the pollen concentration prediction program 108 has identified the target plant as being within a growth cycle associated with pollen production, the start date may be the day at which the plant first entered the growth cycle associated with pollen production, based on the growth cycle and the growth stage of the target plant.

In embodiments, for example where a user is navigating based on the pollen yield prediction, the target location may additionally be a location where the user wishes to navigate, the target date may comprise the current date or a date that the user has selected as the day when the user wishes to navigate to the target location. In embodiments, the target plant may be a species or sub-species of plant which the user has selected for pollen yield prediction, and/or may be plant species or sub-species to which the user is allergic, as evidenced by, for example, pre-provided information in a user profile accessible to pollen concentration prediction program 108, or a user's response entered into a prompt provided by pollen concentration prediction program 108 and requesting information regarding the user's allergies. In embodiments, pollen concentration prediction program 108 may only calculate pollen yield for the species and/or sub-species of plant to which the user is allergic.

At 312, the pollen concentration prediction program 108 may predict a pollen distribution at the multiple locations based on the pollen yield and an aerodynamic model. Here, the aerodynamic model may be a machine learning model which may be trained to model the airborne flow and diffusion of particulates from an origin point, and which provides a particle diffusion coefficient for a target location at a target date given the environmental diffusion conditions around the target location at the target date. The pollen concentration prediction program 108 may accordingly extract environmental diffusion conditions from a variety of real-time IoT sensor data and/or meteorological data including, for example, wind speed and direction, vegetation density, humidity, et cetera. In embodiments, the aerodynamic model may further accept topographical information as input, and pollen concentration prediction program 108 may extract topographical information using locatable images of or taken at or near the location to identify topographic features such as buildings, trees, terrain features, et cetera. The pollen concentration prediction program 108 may provide the extracted environmental diffusion conditions, and in embodiments, topographical information, pertaining to the target location at the target date, to the aerodynamic model as inputs to receive the particle diffusion coefficient as an output. In embodiments, the pollen concentration prediction program 108 may provide environmental diffusion conditions pertaining to a period of time between a start date and the target date as inputs to the aerodynamic model, and may receive as output a particle diffusion coefficient that pertains to the period between the start date and the target date, and represents the average daily rate at which pollen diffuses outwards from the target location in the period of time between the start date and the target date. The pollen concentration prediction program 108 may modify the pollen yield of the target plants by the particle diffusion coefficient to predict a pollen distribution for the target plant, where the pollen distribution represents the actual amount of pollen from each target plant which is present at the target location.

At 314, the pollen concentration prediction program 108 may navigate a user based on the predicted pollen distribution. Here, the pollen concentration prediction program 108 may provide instructions to a user to navigate within the region and through or around at least one target location based on the pollen concentration of target plant species or sub-species at the target location on the target date. The pollen concentration prediction program 108 may display a graphical user interface to the user on the user device 103 or display of UI device set 123 of computer 101; the graphical user interface may, for example, prompt the user to enter a target destination, target time, and one or more target plant species or sub-species to which the user may be allergic or otherwise wish to avoid. The pollen concentration prediction program 108 may then, utilizing an online mapping process, derive a route from the user or from a user-specified starting location to the target location at the target time that avoids locations containing the target plant species or sub-species, and/or avoids locations containing pollen concentrations from the target plant species or sub-species that exceed a threshold value, where the threshold value represents a pollen concentration sufficient to trigger allergic symptoms or allergic symptoms of a particular severity in the user. The threshold value may be pre-provided, entered by the user, and/or derived by a machine learning model based on historical data.

In embodiments, rather than or in addition to navigating the user, the pollen concentration prediction program 108 may provide alerts or notifications to the user when the pollen concentrations of the target plant species at the user's location exceed the threshold value, or at times in the future when the target plant species at the user's location will be predicted to exceed the threshold value.

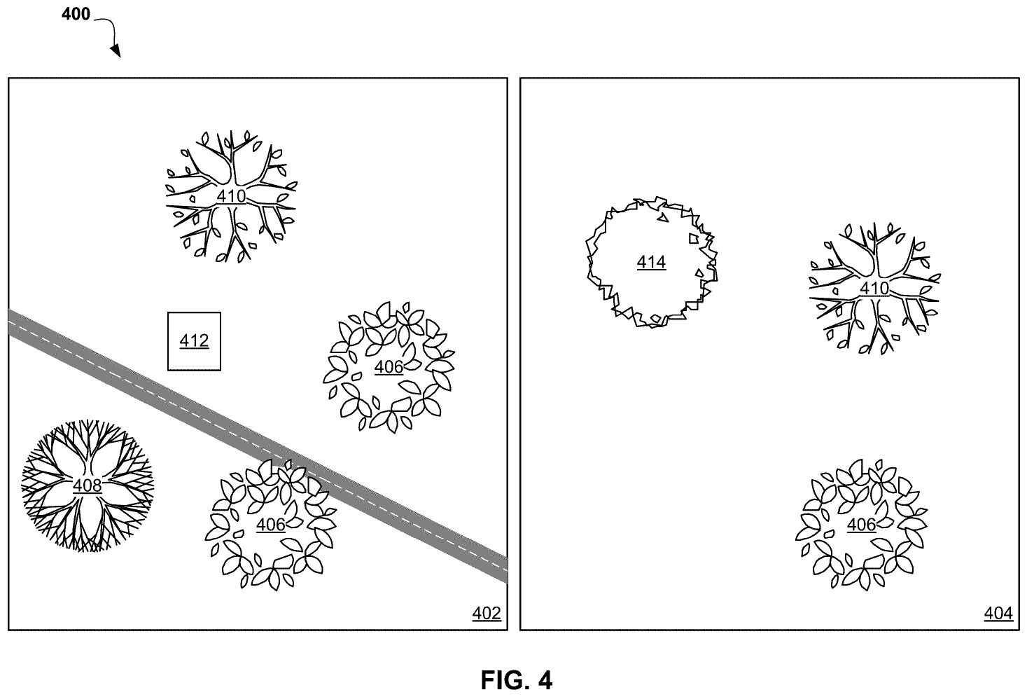

Referring now to FIG. 4, a diagram illustrating an exemplary use case 400 of pollen concentration prediction process 300 is depicted according to at least one embodiment. Here, two separate regions are depicted: a first region 402 and a second region 404. First region 402 comprises four plant species: ash 406, beech 408, and cedar 410. First region 402 also comprises an environmental monitoring station 412, which comprises IoT sensor set 125. Second region 404 comprises three plant species: ash 406, cedar 410, and dogwood 414. The pollen concentration prediction program 108 has, for each species, designated one of the two regions as a reference region. Ash 406 and cedar 410 are present in both regions, and therefore either region may be suitable as a reference region; as such, the pollen concentration prediction program 108 has designated the first region 402 as the reference region for ash 406, and the second region 404 as the reference region for cedar 410. Beech 408 and dogwood 414 are only present in the first region and the second region, respectively. Because the pollen concentration prediction program 108 may only assign a region to a species as a reference region if the species is present in that region, the pollen concentration prediction program 108 designates first region 402 as the reference region for beech 408 and second region 404 as the reference region for dogwood 410.

The pollen concentration prediction program 108 may train four separate growth cycle prediction models, one each for ash 406, beech 408, cedar 410, and dogwood 414. The growth cycle prediction models for ash 406 and beech 408 are trained on plant data, meteorological data, and historical phenological data recorded within their reference region, first region 402; the growth cycle prediction models for cedar 410 and dogwood 414 are trained on plant data, meteorological data, and historical phenological data recorded within their assigned reference region, second region 404.

Because first region 402 is further north than second region 404, first region 402 is colder and experiences less sunlight. As a result, the plant species in first region 402 have only entered into their flowering stage in the past week, whereas the plant species in second region 404 have been flowering for two weeks. The pollen concentration prediction program 108 creates deviation correction models to normalize and correct this discrepancy in growth rates and growth cycles between the regions. Because ash 406 is present in both regions, pollen concentration prediction program 108 may create a deviation correction model for ash 406 pertaining to the second region 404, to normalize the environmental differences between first region 402, the reference region for ash 406, and second region 404, a non-reference region for ash 406. Likewise, the pollen concentration prediction program 108 may create another deviation correction model for beech 408 with respect to first region 402, to normalize differences between second region 404, the reference region for beech 408, and first region 402, a non-reference region for beech 408. Because cedar 410 and dogwood 414 are only present in one region, pollen concentration prediction program 108 does not create deviation correction models for them, as normalization is only necessary to correct discrepancies between regions.

It may be appreciated that FIGS. 2-4 provide only illustrations of individual implementations and do not imply any limitations with regard to how different embodiments may be implemented. Many modifications to the depicted environments may be made based on design and implementation requirements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for pollen prediction, the method comprising:
identifying, by a classification model, one or more plant species within a plurality of locatable images taken at a plurality of locations;
creating one or more growth cycle prediction models for the one or more plant species;
modelling one or more pollen count mappings for the one or more plant species;
predicting one or more pollen yields at the locations for the one or more plant species based on the one or more growth cycle prediction models and the one or more pollen count mappings; and
calculating a pollen distribution at the locations based on the one or more predicted pollen yields and one or more aerodynamic models.

2. The method of claim 1, further comprising:
navigating a user based on the calculated one or more pollen distributions at one or more of the locations.

3. The method of claim 1, wherein the locations are located within a plurality of regions.

4. The method of claim 3, further comprising:
designating one of the regions as a reference region for a plant species of the one or more plant species; and wherein the growth cycle prediction model for the plant species is created based on historical phenological data of the reference region.

5. The method of claim 4, further comprising:
creating one or more deviation correction models for the plant species based on historical phenological data pertaining to the one or more regions comprising the plant species that are not the reference region of the plant species.

6. The method of claim 5, wherein the calculating comprises:
responsive to determining that a location of the locations does not comprise the reference region, normalizing an output of the growth cycle prediction model pertaining to the plant species using a deviation correction model of the one or more deviation correction models pertaining to the plant species and a region of the regions comprising the location.

7. The method of claim 1, wherein the calculating is based on a plurality of real-time Internet of Things sensor data.

8. A computer system for pollen prediction, the computer system comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
identifying, by a classification model, one or more plant species within a plurality of locatable images taken at a plurality of locations;
creating one or more growth cycle prediction models for the one or more plant species;
modelling one or more pollen count mappings for the one or more plant species;
predicting one or more pollen yields at the locations for the one or more plant species based on the one or more growth cycle prediction models and the one or more pollen count mappings; and
calculating a pollen distribution at the locations based on the one or more predicted pollen yields and one or more aerodynamic models.

9. The computer system of claim 8, further comprising:
navigating a user based on the calculated one or more pollen distributions at one or more of the locations.

10. The computer system of claim 8, wherein the locations are located within a plurality of regions.

11. The computer system of claim 10, further comprising:
designating one of the regions as a reference region for a plant species of the one or more plant species; and wherein the growth cycle prediction model for the plant species is created based on historical phenological data of the reference region.

12. The computer system of claim 11, further comprising:
creating one or more deviation correction models for the plant species based on historical phenological data pertaining to the one or more regions comprising the plant species that are not the reference region of the plant species.

13. The computer system of claim 12, wherein the calculating comprises:
responsive to determining that a location of the locations does not comprise the reference region, normalizing an output of the growth cycle prediction model pertaining to the plant species using a deviation correction model of the one or more deviation correction models pertaining to the plant species and a region of the regions comprising the location.

14. The computer system of claim 8, wherein the calculating is based on a plurality of real-time Internet of Things sensor data.

15. A computer program product for pollen prediction, the computer program product comprising:
one or more computer-readable tangible storage medium and program instructions stored on at least one of the one or more tangible storage medium, the program instructions executable by a processor to cause the processor to perform a method comprising:
identifying, by a classification model, one or more plant species within a plurality of locatable images taken at a plurality of locations;
creating one or more growth cycle prediction models for the one or more plant species;
modelling one or more pollen count mappings for the one or more plant species;
predicting one or more pollen yields at the locations for the one or more plant species based on the one or more growth cycle prediction models and the one or more pollen count mappings; and
calculating a pollen distribution at the locations based on the one or more predicted pollen yields and one or more aerodynamic models.

16. The computer program product of claim 15, further comprising:
navigating a user based on the calculated one or more pollen distributions at one or more of the locations.

17. The computer program product of claim 15, wherein the locations are located within a plurality of regions.

18. The computer program product of claim 17, further comprising:
designating one of the regions as a reference region for a plant species of the one or more plant species; and wherein the growth cycle prediction model for the plant species is created based on historical phenological data of the reference region.

19. The computer program product of claim 18, further comprising:
creating one or more deviation correction models for the plant species based on historical phenological data pertaining to the one or more regions comprising the plant species that are not the reference region of the plant species.

20. The computer program product of claim 19, wherein the calculating comprises:
responsive to determining that a location of the locations does not comprise the reference region, normalizing an output of the growth cycle prediction model pertaining to the plant species using a deviation correction model of the one or more deviation correction models pertaining to the plant species and a region of the regions comprising the location.
